(19) United States
(12) Patent Application Publication
(10) Pub. No.: US 2006/0160238 A1
(43) Pub. Date: Jul. 20, 2006

(54) Method and device for analysis, verification and quality assurance of drugs for injection or infusion

(75) Inventor: Bo Lennernas, Uddevalla (SE)

Correspondence Address: YOUNG & THOMPSON, 745 SOUTH 23RD STREET, 2ND FLOOR, ARLINGTON, VA, 22202, US

(73) Assignee: Oncolog Medical QA AB, Uppsala (SE)

(21) Appl. No.: 10/559,186

(22) PCT Filed: Jun. 9, 2004
(86) PCT No.: PCT/SE04/00900

§ 371 Date: Dec. 1, 2005

(30) Foreign Application Priority Data
Jun. 10, 2003 (SE) 0301663-1

(51) Int. Cl. G01N 33/00

(52) U.S. Cl. 436/164

(57) ABSTRACT

The present invention relates to a method and device for non-invasive analysis of the identity and concentration of drugs that are to be administered by injection or infusion.

### FIELD OF THE INVENTION

The present invention relates to a method and device for non-invasive analysis of the identity and concentration of drugs that are to be administered by injection or infusion.

### BACKGROUND OF THE INVENTION

Administration by injection or infusion of drug solutions is performed under medically controlled conditions. The injection or infusion rate and other technical parameters are monitored by the medical personnel and the well-being of the patient is also monitored by regular observations. Records of the diagnosis of the patient's disease and planned treatment thereof are usually stored in the hospital databases where the hospital pharmacy can find the name of the prescribed drug as well as the prescription of the drug specified for each individual patient. The preparation of the drug is ordered by the treating physician for each patient at the time of administration. The drug preparation process is usually performed at the hospital pharmacy. The pharmacy personnel receive the prescription through the hospital database and mix the prescribed drug solution in accordance therewith. The risk for mistakes by the personnel can never be totally avoided since the mixture is made manually. Another weak link in the administration procedure regarding the quality assurance and the safety of the patient is when the prepared drug is transferred from the hospital pharmacy to the location where the administration will take place since many different drug preparations are handled at the same time and by different hospital personnel. There is an obvious risk for mix-up of different medicaments at the moment when the drug container is connected to the device used for administration. In many instances the final preparation of the drug is performed locally at the hospital ward by nurses that have limited pharmaceutical training. Since drugs for injection or infusion often are highly potent, errors in drug composition and concentration have very serious effects on the patient and can even be lethal.

Miscomprehension in drug prescriptions, which can involve poor handwriting, confusion between drugs with similar names, misuse of zeroes and decinal points, confusion of metric and other dosing units, and inappropriate abbreviations is known as one of the most common errors in medication in hospitals.

In the field of analytical methods for chemical solutions different types of spectrophotometric methods and other non-invasive test systems have been used over the years. A wide range of analytical equipment is available on the market providing robust and cost-effective analyses. Methods such as absorption spectrophotometry in the infrared, visible or ultraviolet wavelength range, fluorescence spectrophotometry, Raman spectrophotometry, nuclear magnetic resonance (NMR) as well as electron spin resonance (ESR) are widely used for this purpose. These methods can differentiate between different molecules in a solution by identifying a unique response profile for each kind of molecule and the concentration can be determined by the magnitude of the response. The result of these analyzing techniques provides both qualitative and quantitative information of the analysed solution.

### SUMMARY OF THE INVENTION

As earlier described, there is the risk for administering an incorrect concentration of a prescribed drug or a different drug than the one prescribed, which often has very serious consequences for the patient. A final check and verification of the content in the drug container, by analyzing whether it contains the correct drug or not, and if the concentration of it is as prescribed, would eliminate the severe consequences of any mistake. Therefore, for this purpose a novel application of known analytical methods has been developed.

In a first aspect, the invention as defined in claim 1 comprises a method to perform verification and quality assurance of a drug to be administered by injection or infusion comprising
(a) providing the drug to be administered to the administration container,
(b) loading the drug to the analysing unit,
(c) non-invasively determining a value of at least one chemical and/or physical property of a drug solution to generate a profile for the drug,
(d) comparing the obtained profile with a set of known profiles,
(e) if agreement between the obtained profile and the profile of the prescribed drug is reached issuing a message that the treatment is safe and administration of the drug can proceed.

In one embodiment the analytical methods are selected from fluorescence spectrophotometry, Raman spectrophotometry, NMR or ESR.

In a preferred embodiment of the invention the analysis is carried out by absorption spectrophotometry for determination of the drug solution content.

In another embodiment the invention comprises a combination of two or more of these analytical methods.

In another embodiment the invention comprises determination of the identity of a drug solution.

In another embodiment the invention comprises determination of the concentration of the drug solution.

In a further embodiment of the invention a warning message is issued to stop the procedure if an agreement is not reached between the obtained profile and the profile of the prescribed drug.

In a second aspect of the invention there is provided a device for determination of the identity and concentration of a drug to be administered by injection or infusion comprising
(a) an analysing unit containing optical components for analytical determination of at least one property and for issuing a signal corresponding to said property, coupled to,
(b) a drug container or syringe, including a drug solution, and
(c) a central computation unit to which a signal from the analysing unit is transmitted for comparing the generated profile to a set of known profiles, and
(d) a display unit or a printer for displaying the result.

In an optional embodiment an external information network comprising the stored set of known profiles in an external database to which the obtained profile is compared, coupled to the central computation unit.

In a second optional embodiment the central computation unit is coupled to a patient treatment recording system.

In a third optional embodiment the central computation unit is coupled to a treatment planning system.

In another embodiment of the invention the analysing unit comprises an absorption spectrophotometer or a fluorescence spectrophotometer or a Raman spectrophotometer or NMR or ESR or any combination thereof.

### BRIEF DESCRIPTION OF THE DRAWINGS

FIG. 1. Illustrates one embodiment of a system according to the invention.

FIG. 2. Illustrates an alternative embodiment of the invention.

FIG. 3. Examples of spectral profiles for different drugs for infusion or injection.

FIG. 4. Illustrates an analytical unit using light detection.

FIG. 5. Illustrates an alternative analytical unit using NMR and/or ESR.

### DETAILED DESCRIPTION OF THE INVENTION

For the purpose of this application the term “profile” is taken to mean as the spectrum drawn up in a graph as the result of an analytical test run for a certain drug.

The term “data base” comprises for the purpose of this application Electronic Patient Record Systems and Chemotherapy Management Systems.

The inventors have surprisingly found that it is possible to use a non-invasive determination of the chemical profile of a drug for quantitative and qualitative quality assurance and verification in connection with administration of drugs in liquid form.

The method according to the invention is based on a non-invasive analysis of the drug solution by spectrophotometric methods such as absorption spectrophotometry in the infrared, visible or ultraviolet wavelength range, fluorescence spectrophotometry, Raman spectrophotometry, NMR and ESR, including Fourier transforms or other similar methods. These methods can differentiate between different molecules in a solution by identifying a unique response profile for each kind of molecule. The concentration of the component(s) is/are determined by measurement of the magnitude of the response to be compared with the magnitude of response of known concentrations for the substance(s) of interest. A set of known profiles comprising information on chemical identity and concentrations for each profile is recorded and stored in a database. These profiles are used as a reference for comparison against the results from a non-invasive analysis of a drug to be administered. A final testing of identity and concentration of the drug to be administered increases the safety for both the patients as well as for the medical personnel. This quality control should be performed at the preparatory stage of the treatment procedure and before the actual administration of the drug to the patient.

The data base unit is connected to an analytical unit such as an absorption spectrophotometer, fluorescence or Raman spectrophotometer, NMR or ESR equipment that has its detection means coupled to the drug delivery system. The analytical unit determines the chemical profile of the content in the drug container and compares it to the profiles stored in the data base. When the determined chemical profile is found among the profiles in the database the concentration of the drug is calculated from the magnitude of the response. The result of the analysis is displayed on the analysing unit, printed as a treatment record and/or transferred to a patient database where all treatment parameters for each patient are stored. If the prescription is available from the hospital databases it can be compared with the result from the analysis and, if the data agree with regard to both type of drug and concentration, the staff is informed that it is safe to deliver this drug to the patient.

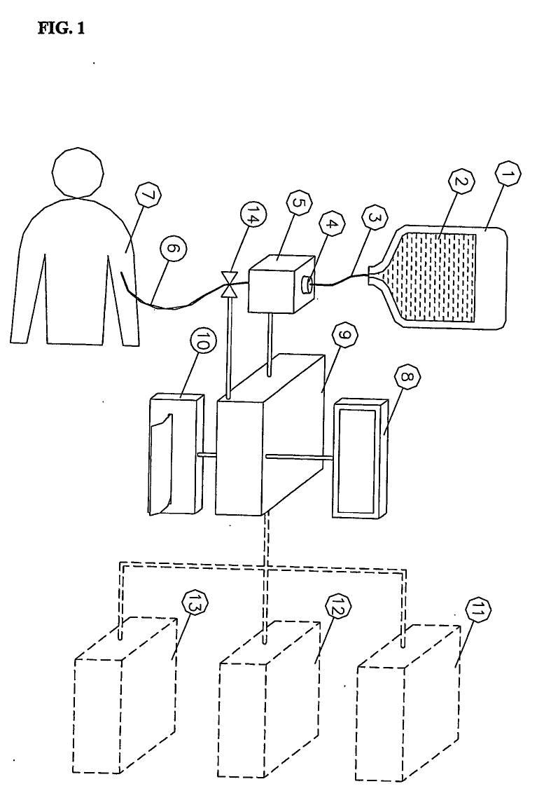

In one preferred embodiment of this invention, as shown in FIG. 1, a drug container (1) contains a sterile drug solution (2) for infusion into a patient. The drug solution flows through a sterile tubing (3) to a sterile analyzing container (4) that is located in the analyzing unit (5). The analyzing unit comprises optical components for absorption spectrum analysis, fluorescence spectrum analysis, Raman spectrum analysis or electromagnetic analysis by NMR or ESR or any combination of these techniques. The signal from the analyzing unit (5) is transmitted to a central computation unit (9) where the measured spectrum, also referred to as profile, is compared to known profiles in a local database. If agreement is found with a previously stored profile for a drug solution the result is presented on a display unit (8) or printed on a printer (10). This system can also be coupled to an external information network where the reference profiles are taken from an optional external database (11) and the result of the analysis is transmitted to a patient treatment management system (12) for recording. The external information network can also supply data about the prescribed treatment for the particular patient from a treatment planning system (13) and if the controlled identity and concentration of the drug solution agrees with the prescription for this patient the system issues a message that the treatment is safe and the infusion process can start. The system may also include a control valve (14) that opens for infusion through the tubing (6) and needle (7) only if the measured profile agrees with the reference profile for the prescribed drug solution for this particular patient.

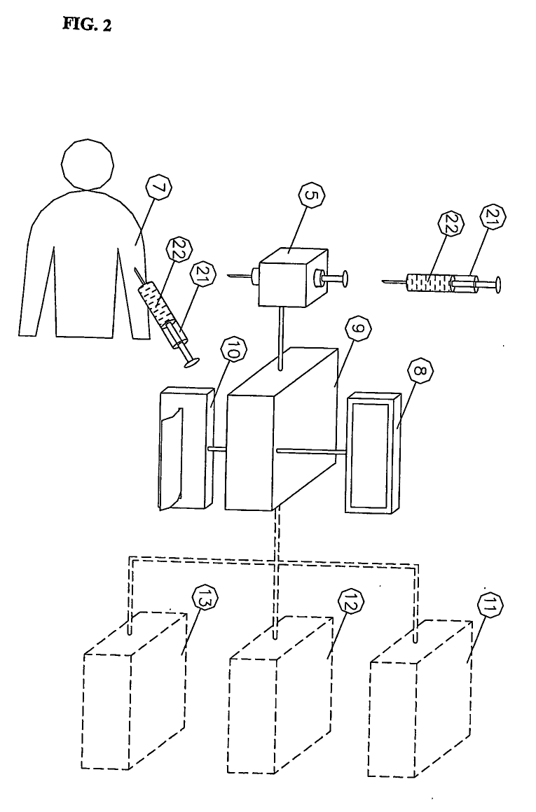

In a second preferred embodiment of this invention, as shown in FIG. 2, a sterile syringe (21) contains a sterile drug solution (22) for injection into a patient. The syringe with the drug solution is placed in the analyzing unit (5). The analyzing unit comprises optical components for absorption spectrum analysis, fluorescence spectrum analysis, Raman spectrum analysis or electromagnetic analysis by NMR or ESR or any combination of these techniques. The signal from the analyzing unit (5) is transmitted to a central computation unit (9) where the measured spectrum, also called profile, is compared to known profiles in a local database. If agreement is found with a previously stored profile for a drug solution the result is presented on a display unit (8) or printed on a printer (10). This system can also be coupled to an external information network where the reference profiles are taken from an optional external database (11) and the result of the analysis is transmitted to a patient treatment management system (12) for recording. The external information network can also supply data about the prescribed treatment for the particular patient from a treatment planning system (13) and if the controlled identity and concentration of the drug solution agrees with the prescription for this patient the system issues a message that the treatment is safe and injection of the drug can proceed.

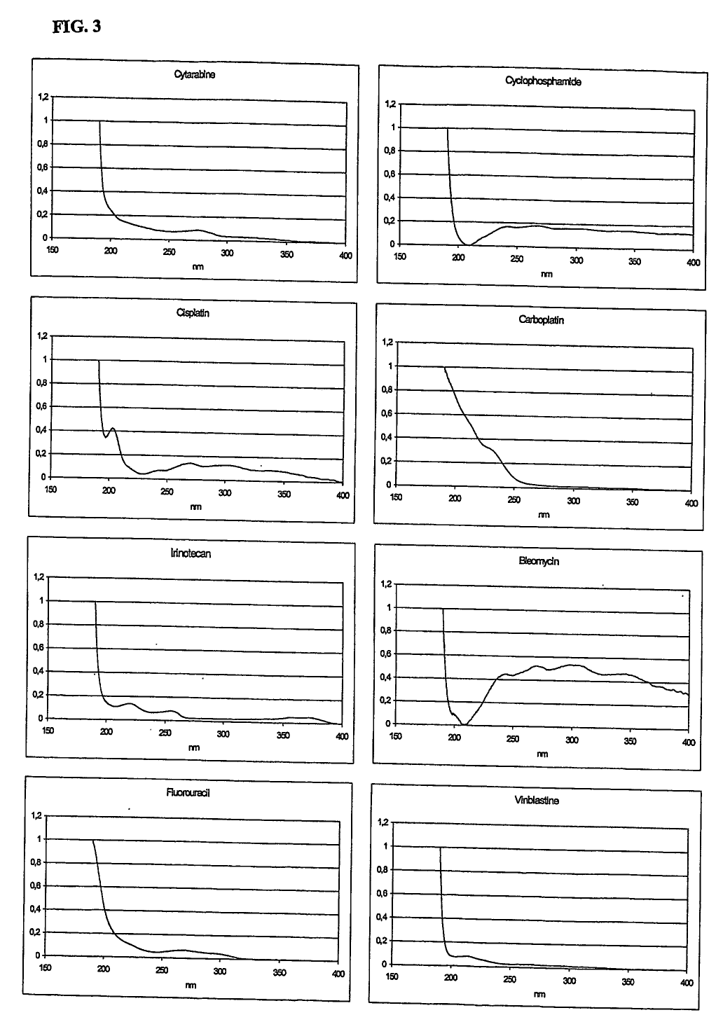

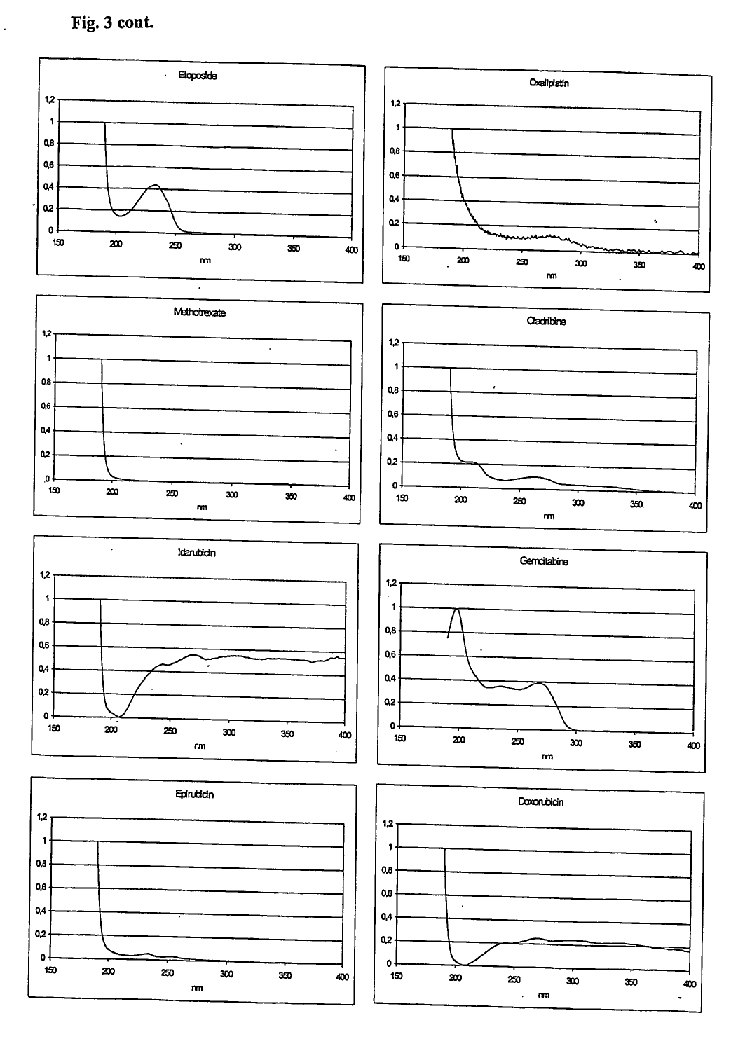

The aim of the following description is to show the unique individual characteristics of drug profiles obtained by absorption spectrophotometry. FIG. 3 shows the absorption spectra in the ultraviolet wavelength range 190-400 nm for a number of commonly used chemotherapy drugs. The illustrated spectra are normalized to the maximum absorption level in order to show the differences in the profiles for the different drug solutions. The profiles show that these drugs have unique profiles that can be clearly separated from each other to identify the type of drug with a high degree of certainty. For applications where the UV spectral profiles of the used drug solutions are too similar to give certain identification, the wavelength range can be extended to the visible and infrared range or complementary techniques such as Raman spectrophotometry, fluorescence spectrophotometry, NMR or ESR can be used.

One important application for this invention is the verification of the drug solution used for chemotherapy against cancer. Here the prescription is tailored for each individual patient and any errors in the type of drug and/or concentration has very serious effects that even can be lethal.

Other potential applications for this invention are intensive care, where nutrition, pain relief and/or medication is given by infusion or injection, dialysis, anaesthesia, surgery, where blood transfusion, blood plasma infusion or blood recovery is used, or any other procedure where drugs or other substances are given to a patient through infusion or injection.

The objective of the following description is to further characterise the analyzing unit of the invention. The analyzing unit (5) in FIGS. 1 and 2 can be designed in many different ways, depending on the choice of analyzing technology or combination of technologies.

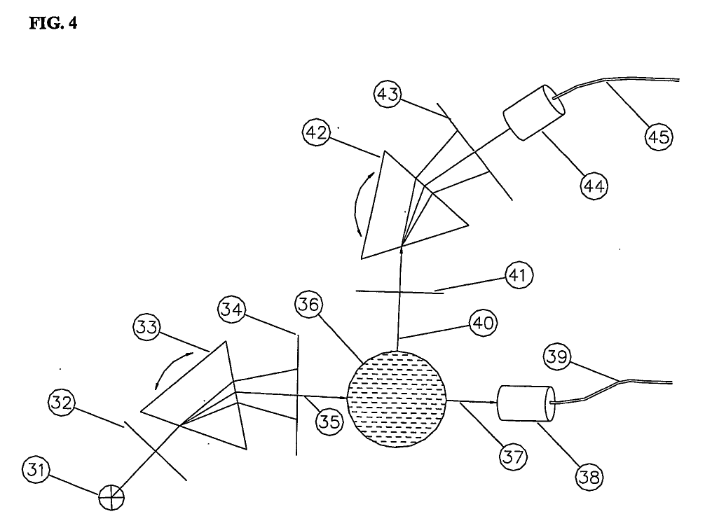

One preferred arrangement for the mentioned optical analyzing techniques is shown in FIG. 4. Light is emitted from a polychrome light source (31), collimated by a slit (32) and separated in wavelengths by a prism or grating (33). The prism or grating (33) is rotated to allow only light with one wavelength at a time to go through the second slit (34) and reach the drug solution in the drug analyzing container (36). The monochrome light (35) is passing straight through the drug solution, where the light absorption is different at different wavelengths. The transmitted light (37) reaches a light detector (38) and the measured light intensity signal is fed through a cable (39) to the electronics unit. The prism or grating (33) is rotated to transmit different wavelengths through the slit (34) and the light absorption in the drug solution as a function of the selected wavelength gives the characteristic absorption spectrum, profile, for the drug. The profile for the drug solution is compared to the known profile for the prescribed drug and if they agree, the type of drug has been verified. The magnitude of the absorption at particular wavelengths gives a direct measure of the concentration by use of the Lambert-Beer law: log Iₒ/I=aₛ*b*c, where Iₒ is the incident light intensity, I is the transmitted light intensity, aₛ is the specific absorbance index for the drug at this particular wavelength, b is the path length of the light through the drug solution and c is the concentration of the solution. Since all the parameters are known the concentration is calculated as c=(log Iₒ/I)/aₛ*b.

A second light detection system that analyzes the fluorescent light from the drug solution can also be used, either alone or in combination with the transmitted light detection system. The fluorescent light (40) is collimated by a slit (41) and separated in wavelengths by a prism or grating (42). The prism or grating (42) is rotated to allow only light with one wavelength at a time to go through the second slit (43) and reach a light detector (44). The signal from the light detector (44) is fed through a cable (45) to the electronics unit. The prism or grating (42) is rotated to transmit different wavelengths through the slit (43) and the fluorescent light emission in the drug solution as a function of the selected wavelength gives the characteristic fluorescence spectrum, profile, for the drug. The profile for the drug solution is compared to the known profile for the prescribed drug and if they agree, the type of drug has been verified. This detection arrangement for fluorescence spectra can also be used to detect Raman spectra.

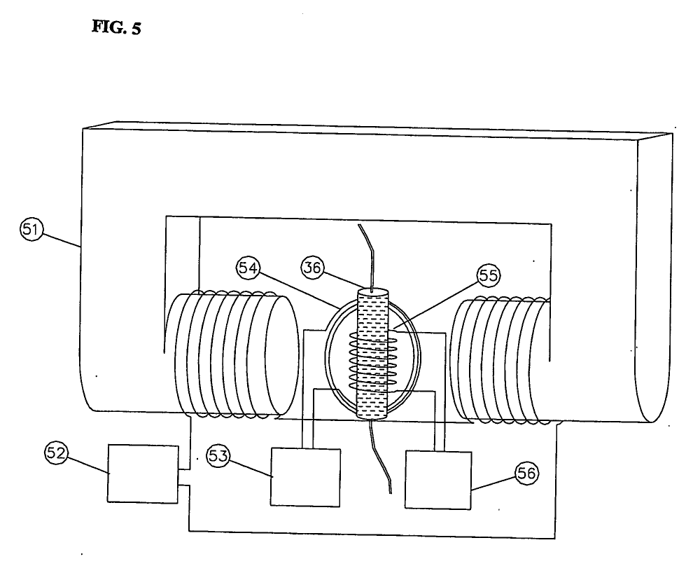

Another preferred arrangement of the analyzing unit (5) is using either NMR or ESR as shown in FIG. 5. In both these techniques the drug solution (36) is placed in a magnetic field, generated by an electromagnet (51) where the magnetic field strength is controlled by a current generator (52). A radiofrequency transmitter (53) energizes a coil (54) that creates an oscillating magnetic field component orthogonal to the field from the electromagnet. When a resonance condition occurs between the nuclear precession frequency and the radiofrequency at a certain magnetic field strength the relaxation energy from the nucleus, as it returns to the lower energy state after the excitation, is picked up in the coil (55) connected to a radiofrequency receiver (56). The received signal as a function of the magnetic field strength gives an NMR spectrum, profile, that is compared with the profile generated by the prescribed drug and if they agree, the type of drug has been verified.

The same system and method is used also for ESR, but in a higher frequency range. These techniques can only verify the type of drug and has no proved method to determine the drug concentration so they are usually combined with the absorption spectrometry concentration measurement described in FIG. 4.

1-12. (canceled)

13. A method to perform verification of correctness and quality assurance of a drug to be administered by injection or infusion comprising
(a) loading the drug to be administered in an administration container,
(b) coupling said administration container to an analysing unit, whereby the administration container and the analysing unit forms a closed system,
(c) in said analysing unit, non-invasively determining by spectroscopic methods one or more values of at least one chemical and/or physical property of the drug solution to generate a profile for the drug,
(d) comparing the obtained profile with a set of known profiles,
(e) if agreement between the obtained profile and the profile of the prescribed drug is reached issuing a message that the treatment is safe and administration of the drug can proceed.

14. The method according to claim 13 wherein the spectroscopic determination is performed by absorption spectrophotometry.

15. The method according to claim 14 wherein the absorption spectrophotometry is performed in a wavelength range selected from ultraviolet, visible and infrared wavelengths, preferably the ultraviolet and visible wavelengths, and more preferably the ultraviolet wavelengths.

16. The method according to claim 13 wherein the spectroscopic determination is performed by fluorescence spectroscopy or Raman spectroscopy or NMR or ESR.

17. The method according to claim 13 wherein the spectroscopic determination is performed by combining ultraviolet absorption spectrophotometry with a spectroscopic method selected from a group of spectroscopic methods consisting of visible absorption spectrophotometry, infrared absorption spectrophotometry, fluorescence spectroscopy, Raman spectroscopy, NMR, and ESR.

18. The method according to claim 13 wherein the spectroscopic determination is for identity measurements of a substance in the drug solution by obtaining a unique profile for each kind of molecule in the drug solution.

19. The method according to claim 13 wherein the spectroscopic determination is for concentration measurements by comparing the magnitude of the obtained profile with the magnitude of known concentration profiles for the substance(s) in the drug solution.

20. The method according to claim 13 wherein if an agreement is not reached between the obtained profile and the profile of the prescribed drug, a warning message is issued to stop the procedure.

21. A device for verification of correctness and quality assurance of a drug to be administered by injection or infusion comprising
a drug container or syringe, including a drug solution coupled to an analysing unit containing optical components for spectroscopic determination of at least one property of said drug solution and for issuing a signal corresponding to said property, said container and said analysing unit forming a closed system,
a central computation unit to which the signal from the analysing unit is transmitted for comparing the generated profile to a set of known profiles, and
a presentation unit for displaying the result.

22. The device according to claim 21, wherein an external information network comprising the stored set of known profiles in an external database to which the obtained profile is compared, coupled to the central computation unit.

23. The device according to claim 21, wherein the central computation unit is coupled to a patient treatment recording system.

24. The device according to claim 21, wherein the central computation unit is coupled to a treatment planning system.

25. The device according to claim 21, wherein the analysing unit comprises an ultraviolet absorption spectrophotometer optionally combined with a visible absorption spectrophotometer or an infrared absorption spectrophotometer or a fluorescence spectroscope or a Raman spectroscope or NMR or ESR.
